(19) United States
(12) Plant Patent Application Publication
(10) Pub. No.: US 2011/0107476 P1
(43) Pub. Date: May 5, 2011

(54) Olive Tree Named 'KADESHON'

(50) ;

(75) Inventors: Shimon Lavee, Rehovot (IL); Benjamin Avidan, Reshon LeZion (IL); Yair Manni, Rosh Haain (IL)

(73) Assignee: STATE OF ISRAEL, MINISTRY OF AGRICULTURE & RURAL DEVELOPMENT, AGRICULTURAL RESEARCH ORGANIZATION

(21) Appl. No.: 12/589,960

(22) Filed: Oct. 30, 2009

(51) Int. Cl. A01H 5/00

(52) U.S. Cl. PLT/158

(57) ABSTRACT

A new and distinct Olea europaea L. olive tree variety named ‘KADESHON’, particularly characterized as a semi-dwarf-sized tree with controlled, medium to weak growth habit; suitable for intensive orchard growing with harvesting from the ground level with only minimal pruning required; resistance to Spilocaea oleagina; and production of large (about 7-8 g), slightly elongated in shape mature fruit, suitable for both green and black pickling.

### LATIN NAME OF THE GENUS AND SPECIES OF THE PLANT CLAIMED

Olea europaea

### VARIETY DENOMINATION

‘KADESHON’

### BACKGROUND OF THE INVENTION

The present invention relates to a new and distinct variety of an olive tree, botanically described as Olea europaea L. of the Oleaceae family, and hereinafter referred to by the variety denomination ‘KADESHON’. The new Olea europaea ‘KADESHON’ is a product of a controlled breeding program conducted by the inventors, Shimon Lavee, Benjamin Avidan and Yair Manni, in Bet Dagan, Israel. The objective of the breeding program was to develop a new Olea europaea variety which is suitable for both green and black pickling, exhibits resistance to Spilocaea oleagina (peacock eye leaf disease), requires minimal pruning and has improved harvesting (due to lower likelihood of damage to mature fruit as result of harvesting).

The new Olea europaea ‘KADESHON’ originated as a result of self pollination of the Olea europaea ‘KADESH’ (unpatented) in 1990 in the controlled breeding program conducted by the inventors in Bet Dagan, Israel. The new Olea europaea ‘KADESHON’ was discovered from the progeny of the stated self-pollination in 1993 and selected in 1994 by the inventors in a controlled environment in Bet Dagan, Israel.

Asexual propagation of the new Olea europaea ‘KADESHON’ by rooting of vegetative cuttings was first performed in the spring of 1995 in Bet Dagan, Israel, and has demonstrated that the combination of characteristics as herein disclosed for the new variety are firmly fixed and retained through successive generations of asexual reproduction. The new variety reproduces true to type.

### BRIEF DESCRIPTION OF THE INVENTION

The following traits have been repeatedly observed and are determined to be unique characteristics of ‘KADESHON’ which in combination distinguish this olive tree as a new and distinct variety:

1. semi-dwarf-sized tree with controlled, medium to weak growth habit;

2. suitable for intensive orchard growing with harvesting from the ground level with only minimal pruning required;

3. resistance to Spilocaea oleagina; and

4. production of large (about 7-8 g), slightly elongated in shape mature fruit, suitable for both green and black pickling.

In comparison to the unpatented, parental variety Olea europaea ‘KADESH’, the new Olea europaea ‘KADESHON’ differs primarily in the traits listed in Table 1.

TABLE 1

| | | Female Parent |
|---|---|---|
| | New Variety | ‘KADESH’ |
| Trait | ‘KADESHON’ | (unpatented) |
| Trunk Diameter | About 46 cm | About 42 cm |
| (measured at 50 cm | | |
| above ground) | | |
| Maximum Tree Height | About 2.2 m | About 4.0 m |

Of the many commercial varieties known to the present inventors, the most similar in comparison to Olea ‘KADESHON’ is Olea europaea ‘BARNEA’ (unpatented). ‘KADESHON’ differs from ‘BARNEA’ in the traits described in. Table 2.

TABLE 2

| Trait | Comparison of Trait of ‘KADESHON’ to ‘BARNEA’ |
|---|---|
| Vigor | Less vigorous than ‘BARNEA’ |
| Tree habit | Less upright and more spreading than ‘BARNEA’ |
| Pruning | Less need of pruning than ‘BARNEA’ |
| Timing of | Fruit matures later than ‘BARNEA’ |
| Mature Fruit | |

### BRIEF DESCRIPTION OF THE PHOTOGRAPHS

The accompanying photographs illustrate the overall appearance of the new Olea europaea ‘KADESHON’ showing the colors as true as is reasonably possible with colored reproductions of this type. Colors in the photographs may differ slightly from the color values cited in the detailed botanical description, which accurately describe the color of ‘KADESHON’.

FIG. 1 shows a side view perspective of a typical 10-year old specimen of ‘KADESHON’ exhibiting its overall appearance.

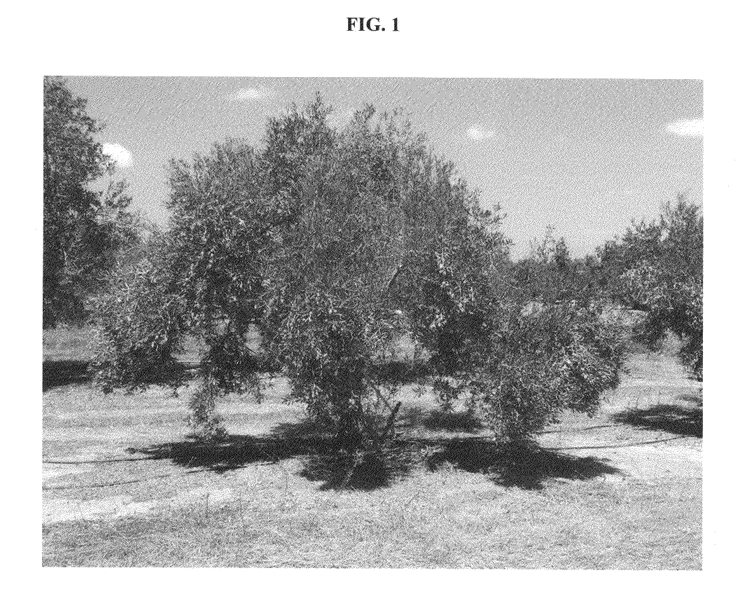

FIG. 2 shows a close-up view of typical unripe, fully grown fruit of ‘KADESHON’

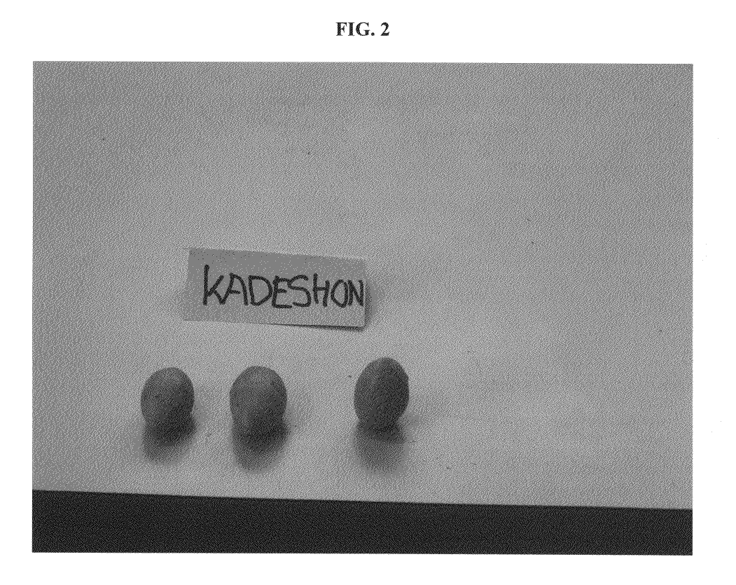

FIG. 3 shows a close-up view of typical ripe, fully grown fruit of ‘KADESHON’ and it's flesh.

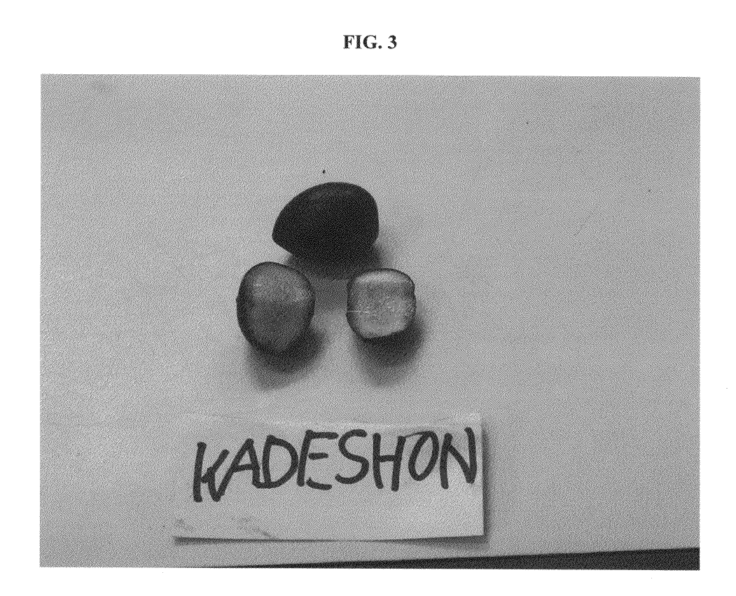

### DETAILED BOTANICAL DESCRIPTION

The new Olea europaea ‘KADESHON’ has not been observed under all possible environmental conditions. The phenotype of the new variety may vary with variations in environment such as temperature, light intensity, and day length without any change in the genotype of the olive tree.

The aforementioned photographs, together with the following observations, measurements and values describe trees of ‘KADESHON’ as grown in the olive farm in Bet Dagan, Israel, under conditions which closely approximate those generally used in commercial practice in Israel. Trees of ‘KADESHON’ are planted at a distance of 4×7 meters in sandy red loam soil at an elevation of about 30 meters above sea level. Trees of ‘KADESHON’ are irrigated by drip system (about 4 liters per hour) during the summer. Average annual rainfall is about 500 mm, with an average 450 mm of rainfall in winter. NPK fertilization (9:3:9.50 ppm) is administered through the drip system. Mean diurnal minimum temperature in January is 7.2° C., and mean diurnal maximum temperature in July is 30.8° C.

Unless otherwise stated, the detailed botanical description includes observations, measurements and values based on four year old ‘KADESHON’ trees grown in the olive farm in Bet Dagan, Israel from 2004 to 2007. Quantitative data are expressed as an average of measurements taken from 10 trees of ‘KADESHON’. The measurements of any individual tree, or any group of trees, of the new variety may vary from the stated average.

Color references are made to The Royal Horticultural Society Colour Chart (R.H.S.), 1986 edition, except where general colors of ordinary significance are used. Color values were taken under conditions of full sunlight in Bet Dagan, Israel.

All of the trees of ‘KADESHON’, insofar as they have been observed, have been identical in all the characteristics described below.
Classification:
   Botanical.—Olea europaea.
Parentage: Self pollination of Olea europaea ‘KADESH’ (unpatented).
Propagation:
   Type.—Vegetative cuttings. Taken from one-year old shoots of juvenile trees (not yet fruit bearing) and which may be produced all year round, or taken from new growth of mature branches of fruit-bearing trees (beginning in spring (April)) and as of July, the mature cuttings are semi wooden and can be propagated throughout the year until February or March.
   Description.—Cuttings of about 12 cm to 15 cm in length, bearing 1 to 2 pairs of top leaves are taken from mother plants (any other plant material is removed). Freshly cut vegetative cuttings are dipped in IBA hormone (2000 ppm) and then planted in peat-perlite substrate (30% peat, 70% perlite). In winter, the propagating bench is heated to 25° C. Mist irrigation is applied for 15 seconds every 20 minutes in the summer, and for 10 seconds every 30 minutes in the winter. In the summer, 70% shading of the cuttings is maintained to keep the temperature below 30° C. In the winter, the temperature is kept above 15° C. Root initiation can be observed after 4 weeks in the summer and 6 weeks in the winter. Two to three weeks later, the cuttings can be hardened.
Growing conditions:
   Light intensities.—Full sunlight.
   Temperature.—Mean diurnal minimum temperature in January is 7.2° C., and mean diurnal maximum temperature in July is 30.8° C.
   Fertilization.—NPK fertilization (9:3:9.50 ppm) is administered through the, drip system.
   Growth regulators.—No growth regulators are used.
   Pruning or trimming requirements.—The olive tree of ‘KADESHON’ as described is grown without training. When the trees are freely grown, the size and the shape assumed by the plants are not typical of Olea europaea L. species.
Tree:
   Age.—Observed trees were 4 years old.
   Vigor.—Medium.
   Crown density.—Medium.
   Height.—About 2.2 meters.
   Attitude of branches.—Erect to spreading.
   Abnormal leaves.—Present; falsiform.
   Trunk.—Diameter (measured at 50 cm above ground): About 46 cm.
   Branches.—Pubescence: Few feathers Color of New Growth (fruiting shoot): Light grey Internode length: Very short to short, about 12 mm to 23 mm.
Leaves:
   Arrangement.—The arrangement of the leaves is typical of Olea europaea L. species (two opposite leaves per each node).
Lamina:
   Overall size.—Small.
   Length.—From 56 mm to 65 mm.
   Width.—From 7 mm to 10 mm.
   Ratio (length/width).—Medium to large.
   Twisting.—Absent.
   Curvature of longitudinal axis of blade.—Flat.
   Overall shape.—Elliptic.
   Apex shape.—Acute.
   Base shape.—Acute.
   Undulation of margin.—Weak.
   Leaf glossiness (both surfaces).—Absent.
   Color (mature).—Upper side: Yellow-green, RHS 146A Lower side: Yellow-green, RHS 148D.
   Petiole.—Length: About 5 mm to 6 mm.
Flowers:
   Blooming time.—Mid-April in Bet Dagan, Israel.
   Ripening time.—Late.
   Structure.—Short and compact.
   Branching.—Strong.
   Axillary flowers.—Absent.
   Flower.—Diameter: About 6 mm to 8 mm Color: White, RHS 155B Pollen color: Yellow, RHS 5B.
   Buds.—Shape: Obovate. Size (just before opening): Small to medium Color: Light yellow-green, RHS 145A Pubescence: Strong.
   Petals.—Number per flower: Four.
   Sepals.—Number per flower: Four.
   Pedicel.—Length: About 1 mm.
Fruit (drupe): In the following description, Position A refers to the position in which the fruit shows its largest asymmetry. Position B can be reached from position A by turning 90 degrees along the longitudinal axes in a way that presents the most developed part of the fruit to the observer (according to UPOV rules).
   Maturity when described.—Ripe for eating.
   Maturity (ripening) period after full bloom.—Late.
   Drupe detachment resistance.—Medium (251-500 g).
   Stalk.—Length: 16 mm to 25 mm Thickness: About 1 mm Color: Yellow-green, RHS 147C.
   Stalk cavity.—Shape: Elliptic. Width: Narrow. Depth: Shallow.
   Overall size.—Medium.
   Length.—About 19 mm to 28 mm.
   Diameter.—About 14 mm to 18 mm.
   Position of maximum diameter.—Central.
   Overall shape.—Elongated.
   Apical shape.—In Position A: Pointed. In Position B: Pointed.
   Base shape.—In Position A: Truncate. In Position B: Rounded.
   Symmetry.—In Position A: Weakly asymmetric. In Position B: Weakly asymmetric.
   Transversal section shape.—Circular.
   Colour.—Unripe (immature): Yellow-green, RHS 144A Ripe (mature): Greyed-purple, RHS 187A Flesh (mature, freshly cut): Light yellow-green, RHS 145B.
   Conspicuous of marbling.—Medium.
   Mucron.—Present.
   Mottles.—Size: Medium Color: Yellow-green, RHS 144C.
   Pistil scar position.—Not central.
Stone (endocarp):
   Length.—About 15 mm to 20 mm.
   Diameter.—About 7 mm to 8 mm.
   Color (when dry).—Beige, closest to greyed-orange group, RHS 164D.
   Overall shape.—In Position A: Oblong to elliptic. In Position B: Oblong to elliptic.
   Distal shape.—In Position A: Pointed. In Position B: Pointed.
   Base shape.—In Position A: Truncate. In Position B: Truncate.
   Symmetry.—In Position A: Symmetrical. In Position B: Symmetrical.
   Shape of cross section.—Circular.
   Position of largest cross section.—Central.
   Grooving.—Medium.
   Groove distribution at apex.—Present.
   Groove distribution at base.—Regular.
   Number of grooves at the base.—About 7 to 10.
   Mucron.—Present.
   Suture conspicuousness.—Absent or very weak.
Resistance to abiotic factors:
   Cold.—Medium-high.
Resistance to parasites:
   Spilocaea oleagina.—‘KADESHON’ exhibits resistance.
   Pseudomonas savastanoi.—‘KADESHON’ has not been tested.

What is claimed is:

1. A new and distinct Olea europaea L. olive tree variety named ‘KADESHON’, as illustrated and described herein.
